(19) United States
(12) Patent Application Publication
(10) Pub. No.: US 2018/0317986 A1
(43) Pub. Date: Nov. 8, 2018

(54) Crescentic or Base Wedge Osteotomy Bunionectomy

(71) Applicant: Nvision Biomedical Technologies, Inc., San Antonio, TX (US)

(72) Inventors: Matthew Jackman, Sherman, TX (US); Thomas Zink, San Antonio, TX (US)

(73) Assignee: Nvision Biomedical Technologies, Inc., San Antonio, TX (US)

(21) Appl. No.: 15/971,256

(22) Filed: May 4, 2018

Related U.S. Application Data
(60) Provisional application No. 62/501,983, filed on May 5, 2017.

(51) Int. Cl. A61B 17/80

(52) U.S. Cl. CPC A61B 17/8095; A61B 17/8047; A61B 17/809; A61B 17/8061

(57) ABSTRACT

A bunion correcting medical device is disclosed that does not require the removal of bone to perform the correction. The device provides the surgeon with the ability to correct an undesirable Inter Metatarsal (IM) angle, and to tailor the correction to the specific needs of a particular patient. Further, the bunion correcting medical device comprises a plate that is distal to the tibialis anterior tendon which provides for a more stable fixation and prevents narrowing of the bone, thereby providing a stronger repair. Thus, the bunion correcting medical device provides for more accurate and precise corrections, and the patient is able to bear weight on the affected bone structure.

### CROSS-REFERENCE TO RELATED APPLICATION

This application claims priority from Provisional Patent Application Ser. No. 62/501,983 filed on May 5, 2017.

### BACKGROUND

The present invention relates generally to a medical device for the foot. More particularly, the present disclosure relates to a bunion correcting medical device which provides minimal loss of bone and/or toe length, preserves the joint, and eliminates bone and joint stiffness, as well as a number of tools for use therewith.

A bunion (hallux valgus) is a deformity of the base joint of the big toe of an individual. The cause of bunions is not clear in many cases, but some believe bunions to have an inherited component. It has also been suggested that wearing shoes with elevated heels and/or a narrow toe-box may contribute to bunion development, as can having flat feet.

The bunion deformity may also cause the foot to rub on shoes or other footwear which may, in turn, result in inflammation and pain. Further, because a bunion occurs at a joint, where the toe bends during normal walking or running movements, the entire body weight of the individual with the bunion rests on the bunion at each step, which can be extremely painful for the individual. Bunions are also vulnerable to excess pressure and friction from shoes, footwear and the like, which can also lead to the development of calluses and/or blisters and, eventually, potential infection.

Typically, in order to correct a bunion, the bone must be cut and straightened and a holding device should be applied to the bone to hold the bone in the straightened position while the bone heals in said straightened position. While traditional methods of correcting bunions have been somewhat effective, said corrective methods don't always correct the bone in the plantar direction and oftentimes result in the loss of bone and/or toe length and do not always result in a stable fixation. This is due, in part, to the removal of bone necessary to perform the bunion correction.

Thus, there exists a long felt need in the art for an improved device that can be used to correct bunions, and that provides for a minimal loss of bone and/or toe length and that preserves the joint, thereby eliminating stiffness. The present invention discloses a bunion correcting medical device that requires little to no removal of bone to perform the correction, and provides the ability to correct an undesirable Inter Metatarsal (IM) angle. Further, the bunion correcting medical device of the present invention comprises a plate that is distal to the tibialis anterior tendon insertion and provides for a more stable fixation. The device of the present invention also prevents narrowing of the bone, thereby providing for a stronger repair. Thus, the bunion correcting medical device provides for more accurate and precise corrections, and the patient is able to bear substantial weight on the affected bone structure following the procedure.

### SUMMARY

The following presents a simplified summary in order to provide a basic understanding of some aspects of the disclosed innovation. This summary is not an extensive overview, and it is not intended to identify key/critical elements or to delineate the scope thereof. Its sole purpose is to present some concepts in a simplified form as a prelude to the more detailed description that is presented later.

The subject matter disclosed and claimed herein, in one aspect thereof, comprises a bunion correcting medical device which provides the ability to correct an inappropriate or undesirable inter metatarsal (IM) angle. The bunion correcting medical device comprises a body portion, a first arm extending outwardly from said body portion and a second arm also extending outwardly from said body portion and positioned at an angle relative to the first arm, such that the first arm extends upwardly from the base plate into a different plane. The base portion and each of first arm and second arm comprise at least one opening or aperture, as explained more fully below. Additionally, the second arm also comprises a stabilizing tab component extending outwardly therefrom to stabilize the device with respect to the bone. The stabilizing tab component of the second arm is positioned at an angle relative to the second arm, and extends upwardly from the second arm into another or different plane.

In a preferred embodiment, the bunion correcting medical device is secured to the bone of a foot with screws, staples, or other fasteners. Specifically, the first arm is first positioned across the width of the affected bone, and the second arm is positioned longitudinally across the length of the same bone. Once in place, the bunion correcting medical device is secured to the bone plantarly with screws or other suitable fasteners. At least two of the above referenced apertures may be threaded to securely receive the fasteners. Further, the screws are inserted dorsally through the bone of the metatarsal and then into the threaded apertures of the bunion correcting medical device plantarly, where they are secured. Additionally, the length of the second arm can be longer than the first arm to suit the needs of a particular patient.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and is intended to include all such aspects and their equivalents. Other advantages and novel features will become apparent from the following detailed description when considered in conjunction with the drawings.

### BRIEF DESCRIPTION OF THE DRAWINGS

FIG. 1A illustrates a front perspective view of the bunion correcting medical device in accordance with the disclosed architecture.

FIG. 1B illustrates a top perspective view of the bunion correcting medical device in accordance with the disclosed architecture.

FIG. 1C illustrates a side perspective view of the bunion correcting medical device in accordance with the disclosed architecture and showing the angle between the first and the second arm.

FIG. 2 illustrates a perspective view of the bunion correcting medical device of the present invention just prior to being secured to a bone in a foot with the use of a guide plate and in accordance with the disclosed architecture.

FIG. 3A illustrates a rear perspective view of the bunion correcting medical device of the present invention attached to the guide plate with a fastener threaded into the base plate in accordance with the disclosed architecture

FIG. 3B illustrates a front perspective view of the bunion correcting medical device of FIG. 3A.

FIG. 4 illustrates a perspective view of one embodiment of an osteotomy guide tool device in accordance with the disclosed architecture.

FIG. 5 illustrates a front perspective view of the osteotomy guide tool device of Claim 4 in accordance with the disclosed architecture.

FIG. 6 illustrates a side perspective view of the osteotomy guide tool device of Claim 4 in accordance with the disclosed architecture.

FIG. 7 illustrates a side perspective view of the osteotomy guide tool device of Claim 4 in operation in accordance with the disclosed architecture.

FIG. 8 illustrates a top perspective view of the bone structure of a bunion in accordance with the disclosed architecture.

FIG. 9 illustrates a top perspective view of the corrected bone structure of a foot in accordance with the disclosed architecture.

FIG. 10 illustrates a top perspective view of the bone structure of a foot with a crescent shaped cut.

FIG. 11 illustrates a top perspective view of the bone structure of a foot with a wedge shaped cut.

FIG. 12 illustrates a perspective view of an alternative osteotomy guide tool device in a locked position in accordance with the disclosed architecture.

FIG. 13 illustrates a perspective view of the osteotomy guide tool device of Claim 12 in an open position in accordance with the disclosed architecture.

### DETAILED DESCRIPTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof.

The present invention discloses a bunion correcting medical device that requires little to no removal of the afflicted bone to perform the correction and provides the ability to straighten an inappropriate or undesirable Inter Metatarsal (IM) angle, and to tailor the amount of correction according to the particular needs of the particular patient. Further, the bunion correcting medical device is positioned distal to the tibialis anterior tendon which provides for a more stable fixation and prevents narrowing of the bone, thereby providing a stronger repair. Thus, the bunion correcting medical device provides for more accurate and precise corrections, and the patient is able to bear weight on the affected bone structure following the use of the bunion correcting medical device. Overall, the bunion correcting device provides quicker healing as the device provides a minimal loss of length of the bone and/or tow, and preserves the joint, thereby reducing the likelihood of joint stiffness.

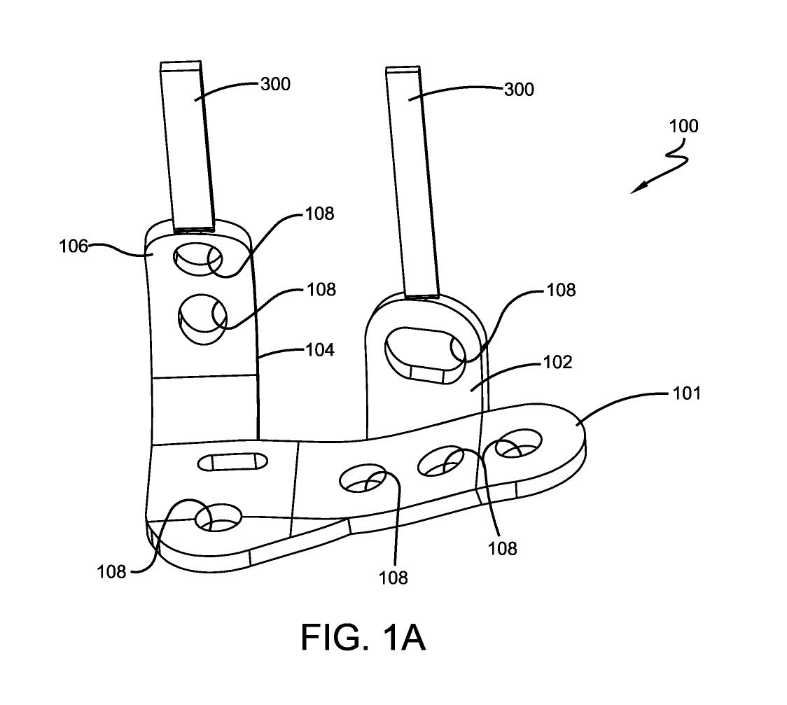

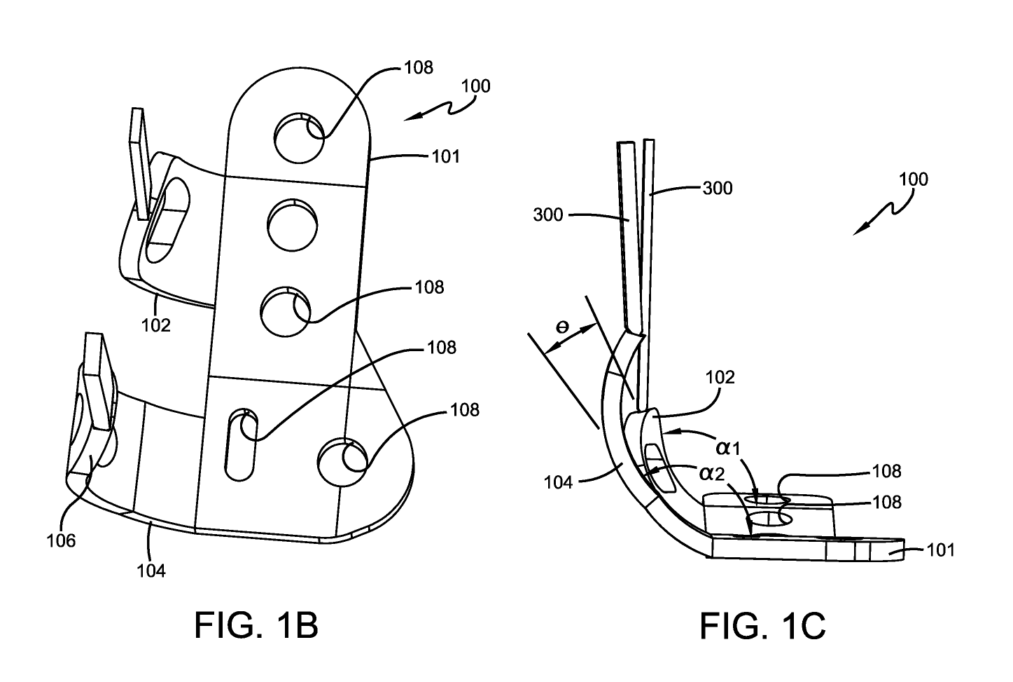

Referring initially to the drawings, FIG. 1A illustrates a front perspective view of a preferred embodiment of the bunion correcting medical device (or holding splint device) 100 of the present invention, and FIG. 1B illustrates a top perspective view of the bunion correcting medical device 100. Bunion correcting medical device 100 preferably comprises a base plate 101, a first arm 102 extending outwardly from said base plate 101, and a second arm 104 that also extends outwardly from base plate 101, and that is positioned at an angle θ relative to first arm 102, such that the first arm 102 extends upward from the base plate in a different plane than the second arm 104. The angle of first arm 102 relative to the second arm 104 is best illustrated in FIG. 1C, which illustrates a side perspective view of the bunion correcting medical device 100.

First arm 102 and the second arm 104 can be any suitable size, shape, and configuration as is known in the art without affecting the overall concept of the invention, though oftentimes the second arm 104 will be longer in length than first arm 102. More specifically, one of ordinary skill in the art will appreciate that the shape and size of the first arm 102 and the second arm 104 as shown in FIGS. 1A-C are for illustrative purposes only and many other shapes and sizes of the first arm 102 and the second arm 104 are well within the scope of the present disclosure. Although dimensions of the first arm 102 and the second arm 104 (i.e., length, width, and height) are important design parameters for good performance, first arm 102 and the second arm 104 may be any shape or size that ensures optimal performance during use. For example, one or both of first arm 102 and second arm 104 may be curved in shape to better accommodate or match the exterior surface of the patient's bone 10.

As best illustrated in FIGS. 1A-C and 3A-B, base plate 101 is a generally plate-like structure for placement under a patient's foot, and each of first arm 102 and second arm 104 extend outwardly from the same side of base plate 101. More specifically, first arm 102 extends outwardly from base plate 101 at an angle α1 of between approximately 30 and 120 degrees relative to base plate 101, and second arm 104 extends outwardly from base plate 101 at an angle α2 of between approximately 30 and 120 degrees relative to base plate 101, though other angles are contemplated to be possible as well.

As best shown in FIGS. 1A and B and 3A and B, base plate 101 comprises at least one, and preferably more than one, continuous openings or apertures 108 formed therein for receipt of a screw or other type of fastener 112, as explained more fully below. Openings 108 are preferably threaded for a more secure connection with fastener 112, but are not required to be.

Further, the base plate 101 of bunion correcting medical device 100 may be contoured for application to the right foot or the left foot depending on the needs and/or wants of a particular patient or surgeon. The bunion correcting medical device 100 allows for adjustability and allows a surgeon to more closely tailor the device to a particular patient's needs. For example, the length of the second arm 104 can be extended as required with respect to the first arm 102, during the manufacturing process so that it is longer in length than that of the first arm 102. Typically, the first arm 102 and the second arm 104 are integrally formed during manufacturing and are typically made of a thin metal, such as titanium, plastic, or other suitable medical material as is known in the art.

Similar to base plate 101, the first arm 102 and the second arm 104 may further comprise a plurality of apertures or openings 108 which extend through the first arm 102 and/or the second arm 104 to accept fasteners 112 such as rivets, screws, staples, or other suitable fasteners known in the art to cooperate to secure the bunion correcting medical device 100 to the patient's bone. Specifically, the first arm 102 and the second arm 104 can accommodate any number of apertures 108 to secure the bunion correcting medical device 100 to the bone, depending on the needs and/or wants of a user, and/or manufacturing constraints. Further, apertures 108 are preferably threaded to secure the fasteners 112, but not all of the apertures 108 need to be threaded. Once in place, the bunion correcting medical device is secured to the bone plantarly with rivets, screws, staples or other suitable fasteners. Further, the fasteners are inserted dorsally through the bone of the metatarsal and then into the threaded apertures 108 of the base plate 101 of the bunion correcting medical device plantarly, where they are secured.

Additionally, the second arm 104 of the bunion correcting medical device 100 comprises a stabilizing tab component (or anti-rotation tab) 106 to stabilize the device 100 with respect to the bone 10. The stabilizing tab component 106 is also positioned at an angle from the second arm 104 and extends upwards from the second arm 104 into another plane. Ideally, the angle of tab component 106 to second arm 104 is between approximately 30 and 120 degrees, though other angles are contemplated to be possible as well. Further, the stabilizing tab component 106 can be any suitable size, shape, and configuration as is known in the art without affecting the overall concept of the invention. One of ordinary skill in the art will appreciate that the shape and size of the stabilizing tab component 106 as shown in FIGS. 1-3 is for illustrative purposes only, and many other shapes and sizes of the stabilizing tab component 106 are well within the scope of the present disclosure. Although dimensions of the stabilizing tab component 106 (i.e., length, width, and height) are important design parameters for good performance, the stabilizing tab component 106 may be any shape or size that ensures optimal performance during use. Typically, the stabilizing tab component 106 is integrally formed with the second arm 106 during manufacturing, and is typically made of a thin metal, such as titanium, plastic, or other suitable material as is known in the art.

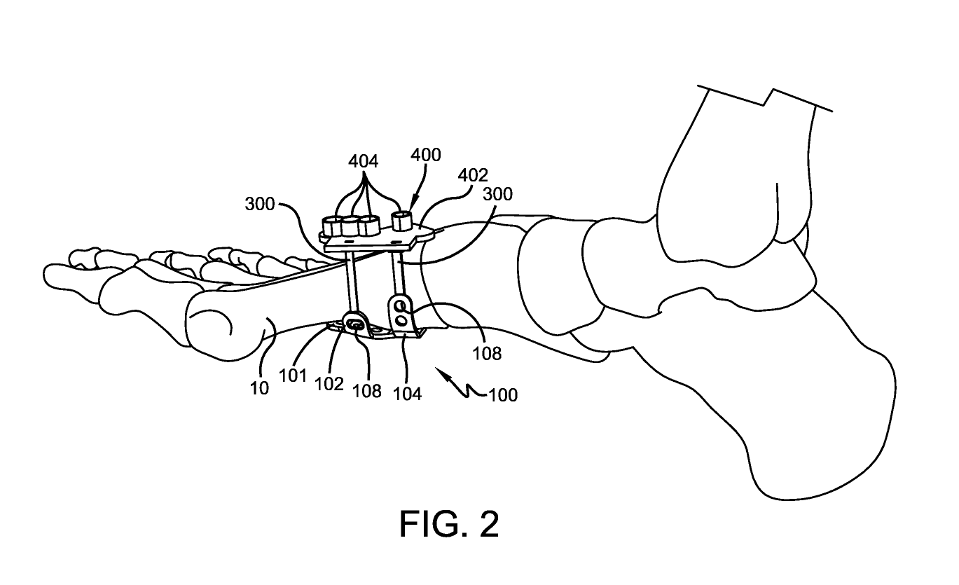

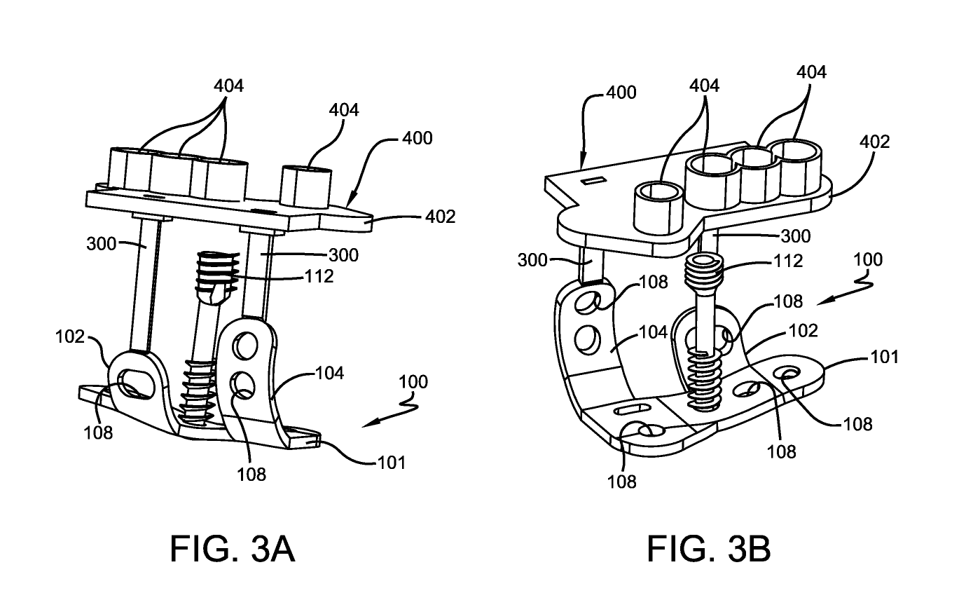

As best shown in FIGS. 1-3, the stabilizing tab component 106 and the first arm 102 may both further comprise a rod-like extension 300, which extends from their respective distal tips to act as a guide for the insertion fasteners 112, such as screws, staples and the like. More specifically and as illustrated in FIGS. 2-3, a guide plate 400 can be positioned on or temporarily attached to the rod-like extensions 300 to further position the trajectory of the fasteners 112 used to mount device 100 to the bone 10. Guide plate 400 is preferably comprised of a base 402 and a plurality of guide cylinders 404 that extend through and rise above base 402, as best illustrated in FIGS. 2-3. Each of guide cylinders 404 is preferably aligned with an opening 108 in base plate 101 so that a fastener 112 can be inserted down through guide cylinder 404, plantarly through bone 10 and secured into threaded opening 108 in base plate 101, which is positioned below the patient's foot, as best shown in FIG. 2. Although dimensions (i.e., length, width, and height) of guide plate 400 and its various components are important design parameters for good performance, guide plate 400 may be any shape or size that ensures optimal performance during use.

Once device 100 is installed in place on bone 10, additional fasteners 112 may be inserted through openings 108 in first arm 102 or second arm 104 to further secure device 100 to bone 10 and/or guide plate 400 can be removed, and rod-like extensions 300 can be broken off or separated from stabilizing tab component 106 and first arm 102 and removed from the patient.

In operation, as shown in FIG. 2, the bunion correcting medical device 100 is applied to the straightened bone 10 of the foot. Specifically, the first arm 102 is first positioned across the width of the bone 10 of the bone, and the base plate 101 is positioned longitudinally across and underneath the length of the same bone 10. The stabilizing tab component 106 is then also positioned across the width of the bone 10 of the foot, distal to the first arm 102. Once in place, the bunion correcting medical device 100 is plantarly secured to the bone 10 with screws, staples, or other suitable fasteners 112 through select apertures 108, which are preferably threaded to receive said fasteners 112 and form a more secure connection between device 100 and the bone 10.

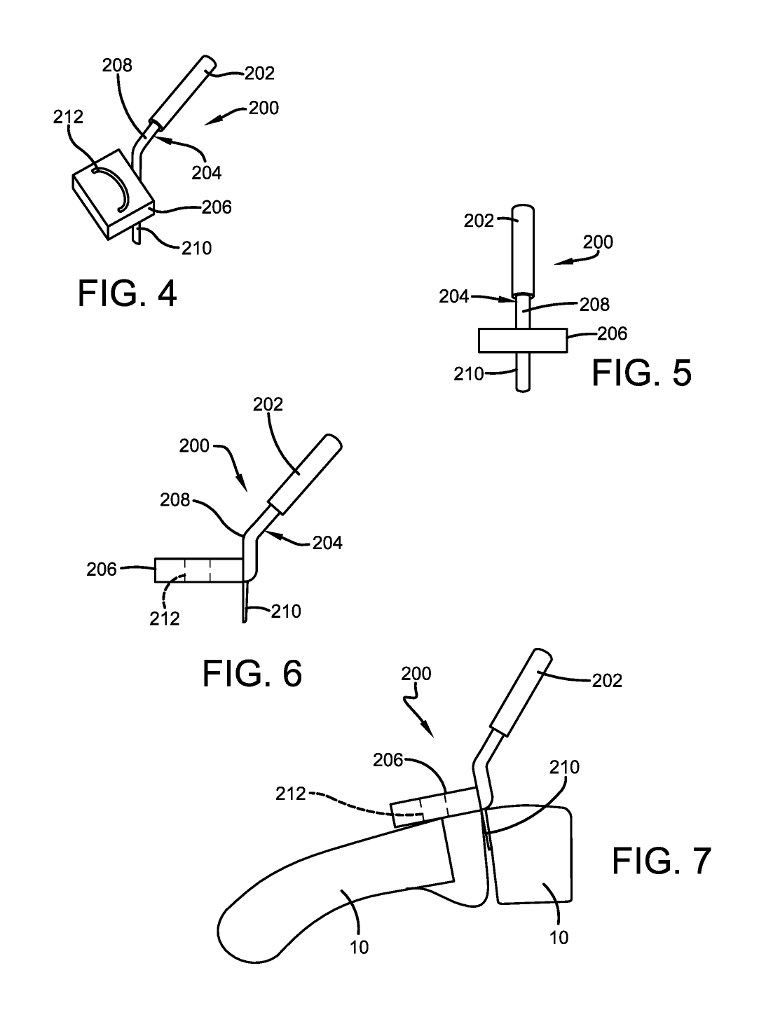

As shown in FIGS. 4-7, an osteotomy guide tool 200 can be used to stabilize the joint of the foot while the bunion correcting medical device 100 is being mounted onto the bone 10 of the patient. The osteotomy guide tool 200 comprises a handle 202, which is connected to a generally L-shaped portion 204, as shown in FIGS. 4 and 6. The L-shaped portion 204 comprises a circular, oval, rectangular, or other suitably shaped cross-section as is known in the art and is typically substantially solid. Further, the L-shaped portion 204 comprises an angled shaft 208 and a vertical shaft 210 which is to be inserted into the joint of the patient, as best shown in FIG. 7. More specifically, the proximal end of the vertical shaft 210 of the L-shaped portion 204 is inserted into the joint of the bone 10 of the patient while the distal end of the angled shaft 208 of the L-shaped portion 204 is connected to the handle 202. A horizontal guide plate portion 206 is connected to the L-shaped portion 204 to be positioned over the bone 10 of the patient in order to control the insertion of the L-shaped portion 204 into the joint of the patient. The horizontal guide plate portion 206 also comprises an aperture 212 which extends through the horizontal guide plate portion 206. The aperture 212 guides the user in cutting the bone of the foot. The aperture 212 can be any suitable shape as is known in the art. Typically, the aperture 212 is crescent shape to make a crescentic cut 12 in bone 10 (as best shown in FIG. 10), or can be wedge shaped to make a closing wedge cut 14 in bone 10 (as best shown in FIG. 11).

In operation, as shown in FIG. 7, the osteotomy guide tool 200 is mounted on the bone 10 of the patient via guide wires (not shown). Further, multiple osteotomy guide tools 200 can be utilized to stabilize the joint of the foot while the bunion correcting medical device 100 is being mounted onto the bone 10 of the patient.

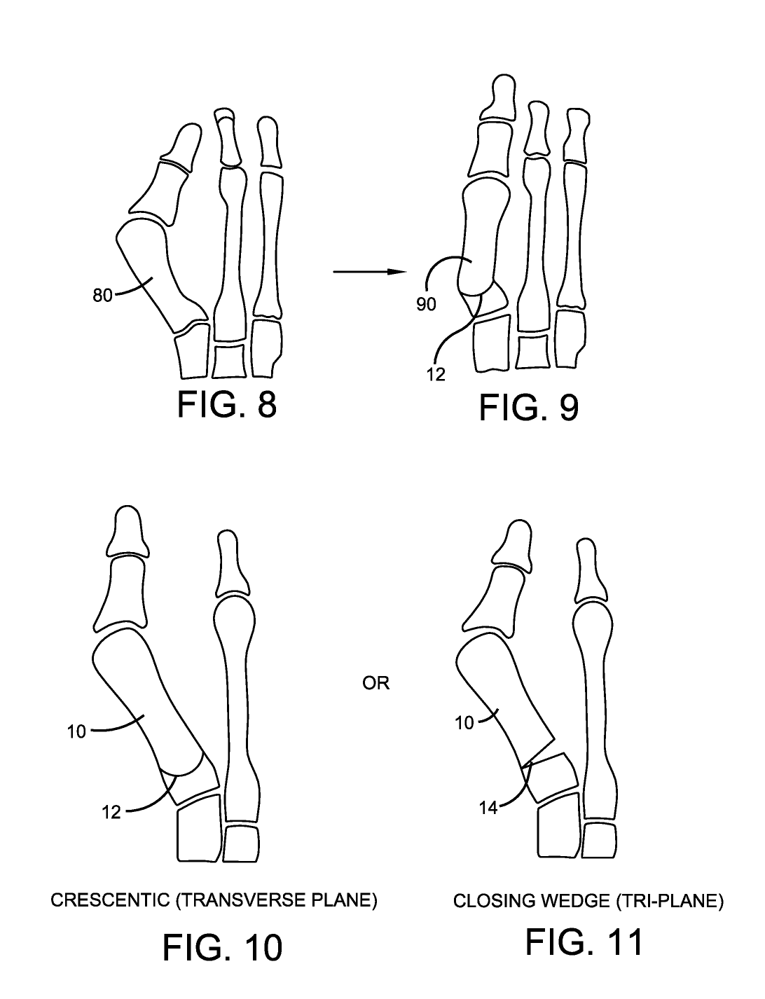

FIG. 8 discloses a bone 80 of the foot of a patient before the application of the bunion correcting medical device 100 and the osteotomy guide tool 200 of the present invention, and FIG. 9 discloses a bone 90 of the foot of a patient after the application of the bunion correcting medical device 100 and the osteotomy guide tool 200. More specifically, FIG. 9 depicts the generally crescent shaped cut 12 in bone 10, which was made using osteotomy guide tool 200 prior to the attachment of bunion correcting medical device 100, which is useful in holding the bone 90 in place as it heals.

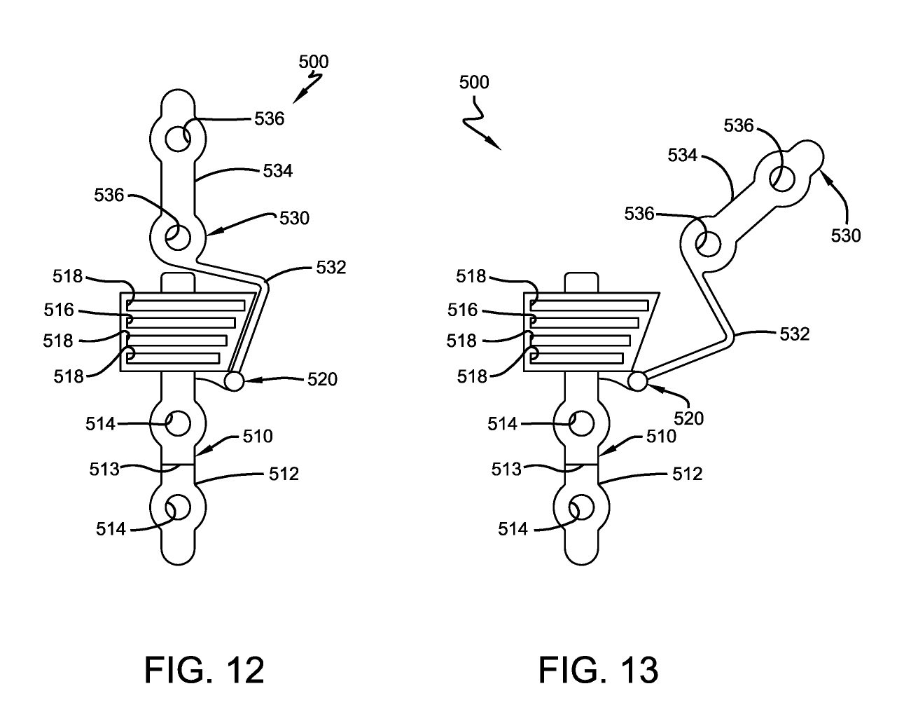

FIG. 12 illustrates a perspective view of an alternative embodiment of an osteotomy guide tool device 500 in a locked position. Guide tool 500 is particularly effective for making a wedge cut 14 on bone 10 and preferably comprises a first portion 510, a locking hinge 520 and a second portion 530. First portion 510 is preferably comprised of a first arm 512, one or more marking 513 on said first arm 512, one or more openings 514 on said first arm 512 and a cut guide plate 516. As best illustrated in FIG. 12, markings 513 are preferably located on the top of first arm 512 and may be, for example, a laser line used to center or align device 500 over the patient's bone joint. Further, openings 514 are useful for removably attaching first portion 510 of device 500 to a patient's bone to be cut through the use of temporary fasteners (not shown).

First arm 512 may be integrally formed with cut guide plate 516, or fixedly attached thereto. Cut guide plate 516 further comprises a plurality of different sized cut apertures 518 that could be used as guides to make wedge-like cuts in a patient's bone in the manner more fully described herein.

Locking hinge 520 may be any hinge type device that can be locked in a particular position, and is attached to and positioned at a desired location on cut guide plate 516, as shown in FIG. 12 by way of an example. Second portion 530 is also attached to said locking hinge 520 such that second portion pivots or rotates about locking hinge 520. Second portion preferably comprises an angled element 532 and a second arm 534 fixedly attached to said angled element 532. Further, like first arm 512, second arm 534 preferably comprises a plurality of openings 536 therein for receipt of temporary fasteners, such as BB tacks (not shown), that can be used to removably attach second portion 530 of device 500 to a patient's bone during the wedge cutting procedure, which is described more fully below.

FIG. 13 illustrates a perspective view of the osteotomy guide tool device 500 of FIG. 12 in an open position, in which second portion 530 is partially rotated about locking hinge 520. During the wedge cutting procedure, first portion 510 and second portion 530 would typically be removably attached to the patient's bone in which the wedge cut will be made with removable fasteners (e.g., BB tacks) and device 500 will be as shown in FIG. 12 while the first cut of the wedge cut is made by inserting a bone cutting device (not shown) into a select one of cut apertures 518 in cut guide plate 516. Following the completion of the first cut, hinge 520 would be unlocked and the second arm 534 rotated into the position shown in FIG. 13, at which time hinge 520 will again be locked, and the second cut of the wedge cut will be made by inserting a bone cutting device (not shown) into a select one of cut apertures 518 in cut guide plate 516.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A bunion correcting medical device which corrects an inter metatarsal (IM) angle of a bone comprising:
a base plate;
a first arm extending outwardly from the base plate at a first angle;
a second arm extending outwardly from the base plate at a second angle that differs from the first angle;
at least one aperture in one or more of the base plate, the first arm and the second arm; and
a stabilizing tab component that extends from a distal end of the second arm.

2. The bunion correcting medical device of claim 1, wherein at least a portion of the first arm is in a different plane than that of the second arm.

3. The bunion correcting medical device of claim 1, wherein the stabilizing tab component extends from the second arm at a third angle.

4. The bunion correcting medical device of claim 1, wherein the first angle is between 30 and 120 degrees relative to the base plate, and the second angle is between 30 and 120 degrees relative to the base plate.

5. The bunion correcting medical device of claim 1, wherein each of the base plate, the first arm and the second arm have an aperture for receipt of a fastener.

6. The bunion correcting medical device of claim 5, wherein at least one of said apertures is threaded.

7. The bunion correcting medical device of claim 1, wherein each of the stabilizing tab component and the first arm comprise a rod-like extension which extends from a distal tip of each of said stabilizing rod component and said first arm.

8. The bunion correcting medical device of claim 1, wherein the stabilizing tab component comprises an opening therein.

9. The bunion correcting medical device of claim 8 wherein the opening is sized to accept a fastener to secure the bunion correcting medical device to the bone.

10. The bunion correcting medical device of claim 1, wherein at least one of the first arm and the second arm is curved.

11. An osteotomy guide tool for making a cut in a bone comprising:
a first portion;
a locking hinge; and
a second portion.

12. The osteotomy guide tool of claim 11, wherein said first portion further comprises a first arm and a cut guide plate.

13. The osteotomy guide tool of claim 12, wherein said first arm comprises at least one opening therein and at least one marking thereon.

14. The osteotomy guide tool of claim 12, wherein said cut guide plate further comprises a plurality of different sized cut apertures therein.

15. The osteotomy guide tool of claim 11, wherein said second portion further comprises an angled element, a second arm with at least one opening therein and further wherein said second portion is pivotably attached to said locking hinge.

16. An osteotomy guide tool for stabilizing a joint of a foot while a bunion correcting medical device is being mounted onto a bone of a patient, comprising:
a handle;
an L-shaped portion which comprising an angled shaft and a vertical shaft to be inserted into the joint of the patient; and
a horizontal guide plate portion connected to the L-shaped portion.

17. The osteotomy guide tool of claim 16 wherein a proximal end of the vertical shaft is inserted into the joint of the bone of the foot and the distal end of the angled shaft is connected to the handle.

18. The osteotomy guide tool of claim 16 wherein the horizontal guide plate portion comprises an aperture which extends through the horizontal guide plate portion.

19. The osteotomy guide tool of claim 18 wherein the aperture is crescent shaped.

20. The osteotomy guide tool of claim 18 wherein the aperture is wedge shaped.
